(19) United States
(12) Patent Application Publication
(10) Pub. No.: US 2025/0262362 A1
(43) Pub. Date: Aug. 21, 2025

(54) CONFIGURATION OF A MEDICAL DEVICE SYSTEM FOR IMPEDANCE-BASED CALIBRATION OF DIALYSIS SESSIONS

(71) Applicant: Medtronic, Inc., Minneapolis, MN (US)

(72) Inventors: Shantanu Sarkar, Roseville, MN (US); Geert Morren, Vissenaken (BE); Juliana E. Pronovici, New Hope, MN (US); Rebecca L. Poindexter, Minneapolis, MN (US); Mirko de Melis, Maastricht (NL); Todd M. Zielinski, McGrath, MN (US); Evan S. Johnson, New Brighton, MN (US); Steven G. Nelson, Raynham, MA (US); John E. Burnes, Blaine, MN (US); Jerry D. Reiland, Coon Rapids, MN (US)

(21) Appl. No.: 18/857,383

(22) PCT Filed: Apr. 14, 2023
(86) PCT No.: PCT/IB2023/053843

§ 371 Date: Oct. 16, 2024

Related U.S. Application Data
(60) Provisional application No. 63/363,457, filed on Apr. 22, 2022.

(51) Int. Cl. A61M 1/16; A61B 5/00; A61B 5/0205; A61B 5/0537; A61B 5/0538; A61B 5/346; A61B 7/04; G16H 40/67

(52) U.S. Cl. CPC A61M 1/1603; A61B 5/0205; A61B 5/0537; A61B 5/0538; A61B 5/7275; G16H 40/67; A61B 5/346; A61B 7/04

(57) ABSTRACT

This disclosure is directed to medical systems and techniques to provide enhanced care to patients by implementing dialysis management. In one example medical system configured for dialytic treatments, a computing device may be configured to perform a risk assessment for a patient having an upcoming dialysis session based on impedance data. In other examples, the computing device may be configured to use the impedance data an accurately estimate fluid volume for controlling an amount of fluid to be removed during the dialysis session.

This application claims the benefit of U.S. Provisional Patent Application Ser. No. 63/363,457, filed Apr. 22, 2022, the entire content of which is incorporated herein by reference.

### FIELD

The disclosure relates generally to medical systems and, more particularly, medical systems configured for use in medical procedures that provide cardiovascular and/or renal care to patients.

### BACKGROUND

Medical professionals may use medical systems on their patients for a number of reasons such as having the medical system record patient data for future use and/or to perform a procedure to treat a health event/malady (e.g., by way of dialysis). Some medical professionals rely on a medical system to provide their patients with the best medical care, and those patients may receive medical care from the medical system at home and/or in a hospital or another healthcare facility. For various purposes, a medical professional may program the medical system to operate as desired, which may be in accordance with a certain algorithm and/or configurable settings. Some example operations use the patient data to calibrate medical system components to detect health events/maladies, deliver a therapy in the form of regular treatments, and so forth.

Medical systems may monitor various types of data of a patient or a group of patients for one or several purposes. Amongst the numerous examples, some medical systems may record measurements of a patient and their heart as indicia of cardiac health for that patient. Other medical systems provide dialysis as a treatment for renal failure including acute kidney failure for a short time until the patient's kidneys improve or for a remainder of the patient's life in cases of chronic or end stage kidney failure. Dialysis refers to a classification of medical procedures to remove urea, toxins, water, and/or electrolytes from the patient's body. Some of these medical systems specialize in performing one or more medical procedures in a dialysis subclassification, such as ultrafiltration, which removes sodium and water from the patient's body. Dialysis, including ultrafiltration, may improve hemodynamics in heart failure (HF) patients.

### SUMMARY

Medical systems and techniques as described herein facilitate procedures corresponding to a dialysis treatment, e.g., for a patient having renal failure or HF. In general, these systems and techniques advantageously use one or more medical devices of the patient as an impedance measuring mechanism to guide a clinician or patient while performing a dialysis session or directing performance of a dialysis sessions by a dialysis system with advantageous reduction or elimination of user intervention.

A variety of medical devices (e.g., implantable devices, wearable devices, etc.) may be configured to monitor and collect data associated with patient physiology and detect changes in patient health that correlate to changes in data recording the patient physiology. Some of the collected data includes impedance data of a fluid (e.g., blood) and/or tissue. The patient's physician may initiate the dialysis session for the patient where a computing device of the dialysis system uses the impedance data as a surrogate for fluid volume information (e.g., measurements). At the very least, the medical system described herein helps physicians with the dialysis session (or another medical procedure to remove unwanted fluid from a body) and in some instances, enables techniques to avoid patient harm (e.g., arrhythmia or death), for example, by assessing/reducing patient risk.

In view of the above, the present disclosure describes a technological improvement and/or a technical solution integrated into at least one practical application because the medical systems and techniques described herein enhance dialytic treatments with informed risk assessment, thereby mitigating or eliminating altogether the problems associated with proceeding with a current set of dialysis parameters, such as when the physician lacks direct impedance measurements of an intravascular space.

The techniques of this disclosure may be implemented by systems including one or more IMDs and that can autonomously and continuously collect physiological parameter data while the IMD is implanted in a patient over months or years and perform numerous operations per second on the data to enable the systems herein to determine risk levels or other data useful for controlling dialysis procedures. Using techniques of this disclosure with an IMD may be advantageous when a physician cannot be continuously present with the patient between dialysis sessions to evaluate the physiological parameters and/or where performing the operations on the data described herein could not practically be performed in the mind of a physician.

In some examples, the techniques and systems of this disclosure may use a machine learning model to more accurately determine a risk level of a dialysis procedure or otherwise infer the patient's condition for determination of parameters of a dialysis procedure based on physiological data collected by an IMD. In some examples, the machine learning model is trained with a set of training instances, where one or more of the training instances comprise data that indicate relationships between various sets of input data and outputs. Because the machine learning model is trained with potentially thousands or millions of training instances, the machine learning model may reduce the amount of error in risk level or other values useful for control of dialysis. Reducing errors using the techniques of this disclosure may provide one or more technical and clinical advantages, such as increasing efficacy of dialysis and patient safety.

In one example, a medical system comprises one or more medical devices configured to collect physiological parameter data of a patient, wherein the physiological parameter data comprises impedance data corresponding to at least one fluid volume in the patient and processing circuitry configured to: determine a dry impedance for the patient having a dialysis session based on the physiological parameter data; and generate output data indicating the dry impedance for presentation to a clinician.

In another example, a medical system comprises one or more medical devices configured to collect physiological parameter data of a patient, wherein the physiological parameter data comprises impedance data corresponding to at least one fluid volume in the patient; and processing circuitry configured to: determine a risk level to the patient from undergoing a dialysis session based on the physiological parameter data; and generate output data indicating the risk level for presentation to a clinician.

In another example, a method performed by processing circuitry of a medical system having one or more medical devices configured to collect physiological parameter data of a patient is described. The method comprises determining a risk level to the patient from undergoing a dialysis session based on the physiological parameter data, wherein the physiological parameter data comprises impedance data corresponding to at least one fluid volume in the patient; and generating output data indicating the risk level for presentation to a clinician.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A illustrates example environment of an example medical system in conjunction with a patient, in accordance with one or more examples of the present disclosure.

FIG. 1B illustrates an example configuration of the example medical device system in FIG. 1A including a plurality of implantable medical devices, in accordance with one or more examples of the present disclosure.

FIG. 2 is a block diagram illustrating an example configuration of the medical device of FIG. 1A, in accordance with one or more examples of the present disclosure.

FIG. 3 is a block diagram illustrating an example configuration of the external device of FIG. 1A, in accordance with one or more examples of the present disclosure.

FIG. 4 is a block diagram illustrating an example system that includes an access point, a network, external computing devices, such as a server, and one or more other computing devices, which may be coupled to the medical device and external device of FIGS. 1-3, in accordance with one or more examples of the present disclosure.

FIG. 5 is a flow diagram illustrating an example operation, in accordance with one or more examples of the present disclosure.

FIG. 6 is a flow diagram illustrating another example operation, in accordance with one or more examples of the present disclosure.

FIG. 7 is an illustration of a chart of dynamic dialysis calibration, in accordance with one or more examples of the present disclosure.

FIG. 8 is a conceptual diagram illustrating an example machine learning model configured to determine one or more of dialysis parameters, target dry weight, fluid volume, or risk of a dialysis session based on impedance measurements.

FIG. 9 is a conceptual diagram illustrating an example training process for an artificial intelligence model, in accordance with examples of the current disclosure.

FIG. 10A is a perspective drawing illustrating an example IMD.

FIG. 10B is a perspective drawing illustrating another example IMD.

Like reference characters denote like elements throughout the description and figures.

### DETAILED DESCRIPTION

In general, medical systems according to this disclosure implement techniques for guiding dialysis sessions based on physiological parameters measured by an implantable medical device (IMD), such as impedance measured by a subcutaneously inserted cardiac monitoring device. In some examples, a medical system performs a risk assessment as part of or in preparation for a dialysis session.

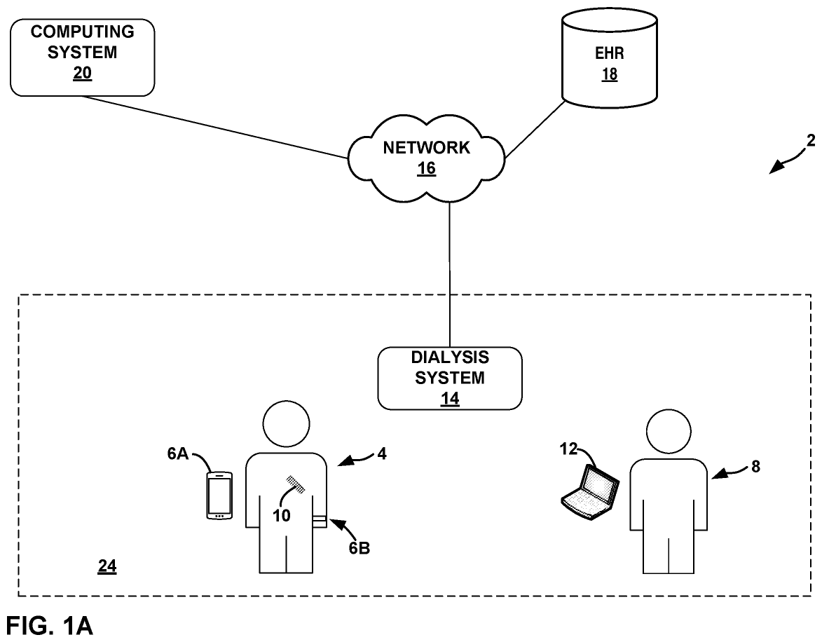

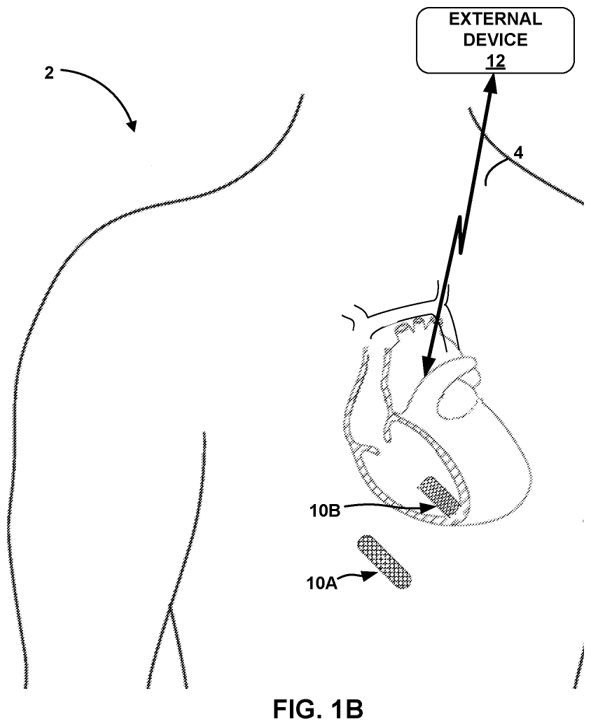

FIG. 1A illustrates the environment of an example medical system 2 in conjunction with patient 4, in accordance with one or more techniques of this disclosure. In addition to one or more patient computing devices 6, such as computing devices 6A and 6B illustrated in FIG. 1A, physician 8 for patient 4 may access data provided by IMD 10 via external device 12. The example techniques may be used with a computing device such as external device 12 or one or more patient computing devices 6, which may be communicably coupled with at least one of IMD 10, dialysis system 14, and/or other devices not pictured in FIG. 1A. Computing devices 6 and 12 may also be communicatively coupled with at least one of Electronic Health Record (EHR) 18 and/or computing system 20 via network 16.

IMD 10, which may be in wireless communication with at least one external computing device and/or other devices not pictured in FIG. 1A, represents one example medical device to be inserted into patient 4 by their physician 8. In some examples, IMD 10 is implanted outside of a thoracic cavity of patient 4 (e.g., subcutaneously in the pectoral location illustrated in FIG. 1A). IMD 10 may be positioned near the sternum or just below the level of the heart of patient 4, e.g., at least partially within the cardiac silhouette. IMD 10 includes a plurality of electrodes (not shown in FIG. 1A) and is configured to sense a cardiac EGM via the plurality of electrodes. In some examples, IMD 10 takes the form of the Reveal LINQ™ or LINQ II™ Insertable Cardiac Monitor (ICM) available from Medtronic, Inc. of Minneapolis, MN.

External device 12 (which may be known as a physician device) may be a computing device with a display viewable by the user and an interface for receiving user input to external device 12. In some examples, external device 12 may be a notebook computer, tablet computer, workstation, one or more servers, cellular phone, personal digital assistant, or another computing device that may run an application that enables the computing device to interact with IMD 10 and/or dialysis system 14.

External device 12 is configured to communicate with patient computing devices 6, IMD 10, dialysis system 14, and, optionally, another computing device (not illustrated in FIG. 1A), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

External device 12 may be used to configure device settings and/or operational parameters for dialysis system 14. External device 12 may be specialized device for enabling physician control over dialysis system 14 and its performance of a dialysis session for patient 4, for example, by modifying various ones of such settings and/or parameters as directed by physician 8 (e.g., a programmer for dialysis system 14). In some examples, external device 12 may be an off-the-shelf computing device, e.g., tablet, with specialized software for the techniques described herein.

External device 12 may be used to retrieve data from IMD 10, e.g., directly or via a computing device 6. In some examples, the retrieved data may include sensor data indicative of electrical activity of the heart of patient 4, e.g., electrocardiogram (ECG) data. In some examples, the retrieved data may include values of sensor data including various impedance data including impedance measurements of an intravascular space, an extravascular space, and/or interstitial space. An additional medical device may provide external device 12 with sensor data including impedance measurements. One example of such a device may be an ICM configured to measure impedance for the interstitial space. Additional details for the ICM are provided in U.S. Patent Application Publication No. 2017/0273589 (filed on Mar. 25, 2016 and assigned U.S. patent application Ser. No. 15/081,216), the entire content of which is incorporated herein by reference.

External device 12 may be used to determine a net filtration rate based on impedance measurements for the interstitial space and impedance measurements for an intravascular space for an inter-dialytic period between the dialysis session and a prior dialysis session and/or an intra-dialytic period during the dialysis session. In other examples, external device 12 may be used to determine a net filtration rate based on impedance measurements for the interstitial space and/or the physiological parameter data of another physiological parameter indicative of fluid volume in the intravascular space. The other physiological parameter includes at least one of an electrocardiogram morphology parameter or a heart sound parameter. External device 12 may be configured to output the net filtration rate for presentation to the clinician upon determining a risk level based on the net filtration rate and/or generate dialysis parameter data for controlling the dialysis session based on net filtration rate.

The sensor data includes sensor signals (e.g., raw sensor data) and/or physiological/diagnostic attributes (e.g., processed sensor data). Some or all of the sensor data may be categorized as either impedance data or non-impedance data. Examples of the non-impedance data may include, but are not limited to, a patient profile/history (e.g., a cardiac arrhythmia history), heart rate data (e.g., heart measurements and heart rate variability (HRV)), patient activity data (e.g., accelerometer data, posture data, and/or the like), patient posture data, medical device history (e.g., logs) of detected health events (e.g., cardiac events such as premature ventricular contractions (PVCs)), and/or the like. As explained in detail further below, some or all examples of the non-impedance data may be input for dialysis system 14, such as a respiration rate, posture data perfusion information, ambulatory blood pressure (ABP) results, potassium estimates, and/or the like. Other examples of non-impedance data may include electrocardiogram data (e.g., time-stamped cardiac EGM segments) recorded by IMD 10.

Processing circuitry of medical system 2, e.g., of one or more devices of computing system 20, IMD 10, computing device(s) 6, external device 12, and/or of one or more other computing devices, may be configured to perform the techniques described herein. The processing circuitry of medical system 2 may employ various known mechanisms to capture (e.g., samples) of various examples of the sensor data and generate information representative of fluid volume and, in some cases, physiological effects of changes in fluid volume, over a period of time. Electrodes of IMD 10 may operate as an impedance measuring device for dialysis system 14.

The present disclosure describes a number of techniques for using the impedance data and any of the above-mentioned examples of physiological/diagnostic attributes. In one example, processing circuitry of medical system 2 may be configured to perform a risk assessment for an upcoming dialysis session that patient 4 is scheduled/prepared to undergo. There are a number of techniques for performing the risk assessment in which a probability (e.g., a likelihood) is determined for an adverse event occurring during or as a result of the upcoming dialysis session. Processing circuitry of medical system 2 may perform an example technique implementing machine learning/artificial intelligence mechanisms to determine the above probability. To illustrate by way of example, processing circuitry of medical system 2 may generate a machine learning model by training a multi-variate mathematical function or a probability distribution to predict a likelihood of an adverse outcome from the upcoming dialysis session. One or more model parameters may correspond to fluid volume computations based on impedance measurements and/or other physiological parameters. In other examples, the machine learning model may be configured to determine initial or updated dialysis parameters for dialysis system as described herein.

Dialysis system 14 is described herein as being medical equipment used by physician 8 to provide patient 4 with dialysis therapy consisting of regimented dialysis sessions for either a limited period of time or for a remainder of patient 4's life. In one example, dialysis system 14 may execute steps of a medical procedure to remove unwanted/excess fluid (e.g., fluid constituents such as water, sodium, potassium, and/or the like) and toxins from the body (e.g., blood) of patient 4 in performance of an example dialysis session. Dialysis system 14, in further performance of the example dialysis session may perform one or more steps to return remaining fluid constituents (e.g., of the blood) back into the body of patient 4. As part of the medical procedure, dialysis system 14 may exchange information with external device 12 and for instance, provide additional physiological/diagnostic attributes such as current body weight, blood pressure, other safety measures. Given the nature of the medical procedure, area 24 may represent a medical facility (e.g., a clinic or a hospital) in which patients similar to patient 4 routinely undergo dialysis sessions. Alternatively, area 24 may represent a home or residence of patient 4.

External device 12 may retrieve the sensor data to perform a risk assessment for patient 4 and/or to determine intra-dialytic fluid volume estimate(s) and/or inter-dialytic fluid volume estimates using impedance data (e.g., measurements) recorded during an inter-dialytic period between dialysis sessions (which may be known as dry impedance and/or wet impedance, respectively).

External device 12 may retrieve non-impedance data including posture data from IMD 10 and, perhaps, other devices attached to or inserted into patient 4. In some examples, external device 12 may retrieve the posture data and incorporate that data into impedance fluid monitoring functionality. Having information (e.g., the non-impedance data) at or around a time of recording the impedance data may enhance the fluid volume estimation (e.g., in terms of accuracy) and/or the risk level assessment by processing circuitry of medical system 2. Such information would include posture data, for example, indicating what posture (e.g., a position) patient 4 is in and for what length of time patient 4 has been in that posture when IMD 10 records an impedance measurement. The amount of time a patient is active (e.g., standing) compared to inactive (e.g., lying down) may affect overall fluid distribution, possibly offsetting the recorded impedance measurements, and thus, impact a target dry impedance and/or the risk level described herein.

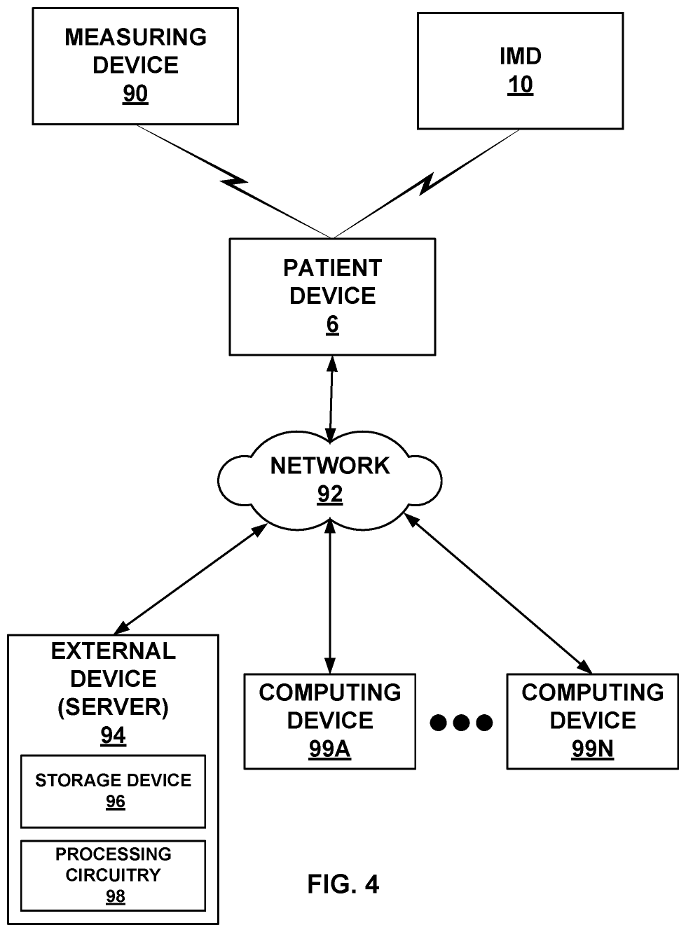

Some examples of IMD 10 provide an option to record (e.g., log) occurrences of any cardiac event, in addition to those that qualify as an arrhythmia, such as QT intervals/changes, repolarization changes, R-wave amplitude and morphology changes, and/or the like. External device 12 may retrieve a log of such events from IMD 10 as part of the non-impedance data. In other examples, external device 12 may be configured to perform specific functionality, such as a programmer for dialysis system 14 and/or IMD 10. As explained in detail in the description of FIG. 4, medical system 2 may involve the programmer calibrating the settings/parameters being programmed in dialysis system 14 and/or IMD 10 based on the impedance data received from IMD 10 (e.g., by way of patient computing device 6A and/or 6B). In some examples, external device 12 may receive some physiological data described herein from computing device 6 that was sensed or collected by that computing device.

Similar to or as part of a memory interrogation of IMD 10, external device 12 may receive impedance data and, in some cases, other physiological data. Processing circuitry and/or dedicated logic circuitry of external device 12 may leverage the received data to improve a risk assessment in preparation of the dialysis sessions. Telemetry hardware (e.g., communication circuitry) of IMD 10 may return the data in response to a request from external device 12. By leveraging the impedance data described herein to continuously monitor fluid status of patient 4, according to one example technique, external device 12 may enable control (e.g., physician control or automated system control) of dialysis parameters (e.g., a blood flow rate, a fluid removal rate, an ultra-filtration rate, a duration, and/or a temperature) during the dialysis session, in effect preventing possible or likely adverse events from occurring and negatively affecting patient 4's health. External device 12 may calibrate one or more dialysis parameters in response to blood volume (BV) changes, dialysate conductivity, urea kinetics and/or thermal energy balance.

Under the control of at least one processor, the telemetry hardware of IMD 10 may receive downlink telemetry from and send uplink telemetry to external device 12 with the aid of an antenna, which may be internal and/or external. There are a number of advantages from providing a physician insights into the dialysis session at any time (e.g., on-demand), adding to the meaningful patient benefits from clinical data for the early detection capabilities of other health events (e.g., heart failure) and enabling the physician (e.g., clinician) to anticipate adverse effects to patient 4 from a dialysis sessions and mitigate/prevent harm to patient through a number of operations.

As will be discussed in greater detail below with respect to FIG. 4, one or more remote computing devices of computing system 20 may interact with IMD 10 in a manner similar to external device 12, e.g., to program IMD 10 and/or retrieve data from IMD 10, via a network. For instance, a remote computing service, which may be similar to that provided by the Medtronic CareLink® Network, communicates with IMD 10 directly over a network connection and/or indirectly through external device 12.

EHR 18 represents a collection of database systems maintained by government agencies and/or healthcare providers and include an electronic version of a patient's medical history, that is maintained by the provider over time, and may include all of the key administrative clinical data relevant to that persons care under a particular provider, including demographics, progress notes, problems, medications, vital signs, past medical history, dialysis run history, recorded vital signs (e.g. weight, blood pressure), immunizations, laboratory data and radiology reports. EHR 18 may provide external device 12 with automated access to any information about patient 4. In some examples, external device 12 may use this information in the above-mentioned risk assessment for an upcoming dialysis session.

In other examples of medical system 2, processing circuitry in a wearable device, such as patient device 6B, may execute same or similar logic as the logic executed by processing circuitry of IMD 10 and/or other processing circuitry as described herein. In this manner, a wearable device or other device may perform some or all of the techniques described herein in the same manner described herein with respect to IMD 10.

FIG. 1B illustrates an example of medical system 2 of FIG. 1A having a first medical device and a second medical device, in accordance with one or more examples of the present disclosure. In some examples, the first medical device may be configured to measure impedance for the interstitial space and the second medical device may be configured to measure impedance for the intravascular space.

In medical system 2, the first medical device (e.g., IMD 10A) and/or the second medical device (e.g., IMD 10B) may be configured to provide (e.g., periodic and/or continuous) impedance measurements (and in some cases measurements of other physiological parameters) to estimate the fluid volume of patient 4 (e.g., blood volume, interstitial volume, and/or a combination of blood volume and interstitial volume). This estimate provides insight into determining an amount of the fluid to remove during an upcoming dialysis session. In this instance, external device 12 may receive the impedance measurements (e.g., which were recorded every hour or every twenty minutes) and by virtue implementing the techniques described herein, these measurements are effectively fluid volume measurements.

Using the fluid volume measurements based on any one or more of intravascular fluid space impedance measurements, extravascular fluid space impedance measurements, and/or interstitial fluid space impedance measurements, external device 12 may generate information (e.g., instructions) directing the dialysis session procedure in its' removal of excess fluid from patient 4. Once removed, patient 4 has a weight (which may be known as dry weight) corresponding to a particular impedance measurement (by IMD 10A and/or IMD 10B) and that impedance measurement may be referred to as dry impedance. In general, the dry impedance is a target impedance (e.g., level) that is determined at an end of the dialysis session, for example, when patient 4 has reached their ideal hydration state. In one example, external device 12 may be used to determine a net filtration rate based on impedance measurements for the interstitial space and impedance measurements for the intravascular space for an inter-dialytic period between the dialysis session and a prior dialysis session and/or an intra-dialytic period during the dialysis session.

FIG. 1B depicts IMD 10A being positioned in a substernal location to record impedance measurements of an extravascular space. These impedance measurements may be used as indirect measurements for fluid volume in an intravascular space. In addition or as an alternative to IMD 10A, a different medical device, such as IMD 10B of FIG. 1B, may be positioned in the intravascular space to directly record impedance and may record intravascular and/or interstitial fluid space impedance measurements. One example of IMD 10B may be an intracardiac pacemaker in a right ventricle of the heart to measure impedance data and/or non-impedance data including heart sounds. IMD 10A may be configured to measure non-impedance data as well, including ECG (R-wave) morphology.

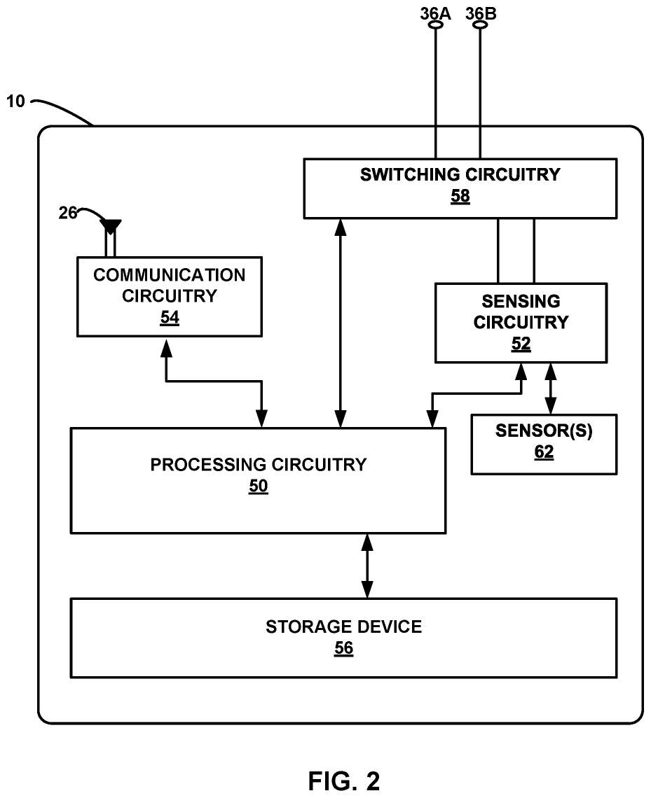

FIG. 2 is a functional block diagram illustrating an example configuration of an IMD 10 of FIGS. 1A and 1B in accordance with one or more techniques described herein.

In the illustrated example, IMD 10 includes electrodes 36A and 36B (collectively “electrodes 36”), antenna 26, processing circuitry 50, sensing circuitry 52, communication circuitry 54, storage device 56, switching circuitry 58, and sensors 62. Although the illustrated example includes two electrodes 36, IMDs including or coupled to more than two electrodes 36 may implement the techniques of this disclosure in some examples. Furthermore, although not illustrated in FIG. 2, IMD 10 may include therapy delivery circuitry or other therapy delivery mechanisms to delivery therapy, e.g., pacing or other stimulation via electrodes 36.

In one example, IMD 10 may record impedance measurements (and, possibly, other diagnostic factors) as part of the recorded/uploaded patient data. IMD 10 may be positioned in one or more locations to allow electrodes 36 to sense impedance over a time period and then, have their respective impedance measurements combined into a fluid volume (e.g., plasma volume) estimate for that time period. As described herein, the fluid volume may be used to control performance of a medical procedure to remove excess or unwanted fluid (e.g., a dialysis session).

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 36 via switching circuitry 58, e.g., to sense electrical activity of the heart of patient 4, for example by selecting electrodes 36 and polarity, referred to as the sensing vector, used to sense the cardiac electrical activity, as controlled by processing circuitry 50. Sensing circuitry 52 may sense signals from electrodes 36, e.g., to produce cardiac EGM data or ECG data as examples of the physiological parameter data. Sensing circuitry 52 may monitor signals from sensors 62 to process various sensor measurements to include as part of the physiological parameter data. Processing circuitry 50 may control one or more of sensors 62 to sense the patient data in some form. Examples of one or more sensors 62 to sense physiological parameter data include any of the following: An accelerometer (e.g., a three-axis accelerometer) to sense patient activity data, posture data, or heart sound data; a pressure sensor; an optical sensor; a gyroscope; a temperature gauge; a moment transducer, and/or the like. Various metrics enable standardized measurement(s) for each sample (e.g., timestamp) of the sensed patient data and differentiation between multiple samples. In some examples, sensing circuitry 52 may include one or more filters and amplifiers for filtering and amplifying signals received from electrodes 36 and/or sensors 62.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth®, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more settings and/or operational parameters of IMD 10 and/or data collected by IMD 10 for transmission to another device using communication circuitry 54. Data stored by storage device 56 and transmitted by communication circuitry 54 to one or more other devices may include the patient data described herein for controlling dialysis session parameters and determining a risk level for a dialysis session.

Sensing circuitry 52 may capture signals from electrodes 36 and/or any one or more of sensors 62, for example, to produce the physiological parameter data for processing by hardware/software (e.g., within IMD 10, external device 12, and/or another device), thereby facilitating (e.g., local or remote) monitoring of fluid volume and patient health (e.g., by an external computing system such as a network-accessible device running a computing service). The above-mentioned hardware/software may include software applications configured with logic to generate additional patient data, for example, data describing a risk level of an adverse outcome associated with a dialysis session.

Sensing circuitry 52 converts to digital form signals corresponding to the sensed electrical or other activity and provides the digitized signals to processing circuitry 50 for analysis by the logic. The logic may direct processing circuitry 50 to apply a pattern recognition technique to interpret electrical activity vectors recorded in corresponding cardiac EGM data as one or more of the above components. In some examples, the waveform may indicate an initial detection of a cardiac event. Processing circuitry 50 generates for display output data indicative of a particular cardiac event type as a classification of the detected cardiac event.

Processing circuitry 50 may execute logic (e.g., software code implementation an algorithm) to determine parameter values for some functionality, including patient health monitoring and health event detection functionality e.g., to determine whether the patient is experiencing a change in health e.g., an increased likelihood of HF, based upon one or more criteria. As described herein, this functionality may be enhanced with monitoring operations configured to warn of or prevent adverse effects related to dialysis.

Processing circuitry 50 and/or sensing circuitry 52 may read/write the patient data from/to storage device 56. Processing circuitry 50 and/or sensing circuitry 52 may cooperate to periodically or continuously record (e.g., monitor) the physiological parameter data (e.g., impedance data). In some examples, processing circuitry 50 and/or sensing circuitry 52 may coordinate to record impedance and other physiological parameter data at a first rate during an inter-dialytic period and a second, higher rate, during an intra-dialytic period. Processing circuitry 50 and/or communication circuitry 54 may upload the patient data via a communication channel (e.g., a Bluetooth® connection) to a computing device, such as patient device 6A of FIG. 1A, external device 12 of FIG. 1A, or a remote device of computing system 20 of FIG. 1A. During an intra-dialytic period, the impedance data (and in some cases other physiological data) may be presented substantially continuously or in substantially real-time as recorded at the higher rate or resolution.

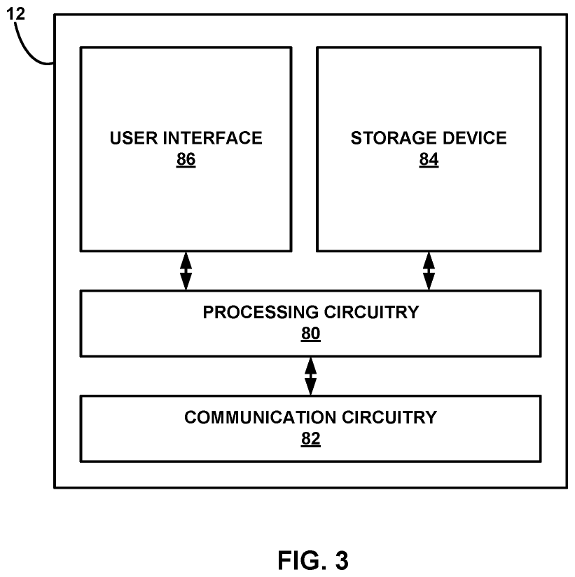

FIG. 3 is a block diagram illustrating an example configuration of components of external device 12. In the example of FIG. 3, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, NFC, RF communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

In some examples, external device 12 and IMD 10 may be Bluetooth® Low Energy (BLE) or Bluetooth-enabled devices and therefore, capable of communicating data to each other, such as the communication, via a radio access technology (RAT), of sensor data comprising impedance measurements. It should be noted that these device(s) may leverage any type of wireless or even a network connection for completing the transmission of the sensor data. IMD 10 may run software code (e.g., of one or more communication protocols) to delineate the sensor data in a message for transmission (e.g., via a wireless connection) as packetized data.

Processing circuitry 80 may transmit an instruction to IMD 10 requesting IMD 10 to export collected data (e.g., cardiac episode data and/or sensor data) to external device 12 (e.g., a memory interrogation). In turn, external device 12 may receive the collected data from IMD 10 and store the collected data in storage device 84. The data external device 12 receives from IMD 10 may include impedance data and other patient data as described herein.

Data exchanged between external device 12 and dialysis system 14 may include device settings and/or operational parameters, as well as the physiological data described herein. External device 12 may transmit data including computer readable instructions which, when implemented by dialysis system 14, may control the dialysis system to set one or more initial settings/parameters and/or change one or more settings/parameters. Processing circuitry 80 may transmit an instruction to update any one or more of the dialysis parameters described herein (e.g., a duration, a frequency, an amount of fluid to remove, and/or the like) based on impedance data and/or other physiological parameters (e.g., dry impedance estimate) and/or risk level.

Processing circuitry 80 may execute logic (e.g., software code implementing an algorithm) to determine parameter values for controlling performance of a dialysis session by dialysis system 14. As described herein, the logic may be configured to advantageously use the impedance data collected by IMD 10. In addition, processing circuitry 80 may also execute logic to determine parameter values for other functionality, including patient health monitoring and health event detection functionality e.g., to determine whether the patient is experiencing a change in health e.g., based upon one or more criteria. As described herein, this functionality may be enhanced with monitoring operations configured to enhance control of dialysis and prevent adverse effects related to dialysis.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to patient 4, a dialysis patient, and electrical activity in or around the heart (e.g., cardiac signals) e.g., a risk level corresponding a likelihood of an adverse effect occurring if patient 4 proceeds with the pending/scheduled dialytic treatment. In another example, processing circuitry 80 may present information indicating new parameter values to update the dialysis system 14. Implementing the updated parameters improves the dialysis session by causing the dialysis system to change the performance of the dialysis session such that it is no longer unsafe to the patient. For example, processing circuitry 80 may update parameters such as dialysis frequency and/or duration (e.g., to increase/decrease a number of dialysis sessions per week, increase/decrease an amount of fluid to remove, and/or increase/reduce the ultrafiltration rate during a dialysis session).

In addition, user interface 86 may include an input mechanism configured to receive input from the user who may be the physician for patient 4. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

The above-mentioned adverse effects may stem from how the physician and/or the dialysis system 14 perform the dialysis session. Conventional dialysis therapy, including intra-dialytic ultrafiltration management, relies on using an estimate of dry weight, which often leads to adverse events like cramping, hypotension, and cardiac arrhythmia. The present disclosure describes techniques directed towards implementing continuous dialytic monitoring and/or calibration for the removal of excess fluid, in either an open loop or closed loop, as part of an innovative dialysis therapy.

Techniques of this disclosure may use impedance as a surrogate of fluid volume and therefore, indicative of an amount of fluid to be removed at an upcoming/next dialysis session. Continuous impedance measurements may be used in intra-dialytic ultrafiltration management to initialize certain parameters for controlling a next dialysis session and then, to update those (e.g., ultrafiltration) parameters (continuously or as needed) for subsequent sessions, thereby reducing a likelihood of any adverse events occurring. Furthermore, processing circuitry 80 may be configured to use other diagnostic measurements (heart rate, cardiac arrhythmia, PVCs, QT changes, repolarization changes, potassium estimates, R-wave amplitude and morphology changes, and/or the like) corresponding to the inter-dialytic period to either setup the initial parameters or customize those parameters for the next dialysis session(s).

FIG. 4 is a block diagram illustrating an example system that includes a plurality of computing devices for dynamic dialysis calibration as describe herein. Measuring device 90 represents any conceivable mechanism for sensing impedance and/or recording impedance measurements and may be an example of, combined with, or used as an alternate to IMD 10 for providing the impedance measurements to other computing device(s). Measuring device 90 may be configured to record impedance measurements and/or other physiological parameters. In addition to patient device 6, any of the other computing devices depicted in FIG. 4 may be configured to receive/send the impedance measurements, including server 94 and remote computing devices 99A-99N (collectively, “computing devices 99”). In the example of FIG. 4, patient device 6, server 94, and computing devices 99 are interconnected and may communicate with each other through network 92.

In the example illustrated by FIG. 4, server 94 includes a storage device 96, e.g., to store data retrieved from IMD 10, and processing circuitry 98. In one example, server 94 may be an example or a part of computing system 20 of FIG. 1A and one or more of computing devices 99 may be external device 12 of FIG. 1A. Alternatively, server 94 may operate in a manner similar to external device 12, such as when patient 4 is at home after undergoing a previous dialysis session (e.g., inter-dialytic period). Although not illustrated in FIG. 4, computing devices 99 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuitry of computing devices 99 may implement any of the techniques described herein to analyze information received from IMD 10, e.g., to determine whether the health status of a patient has changed, to determine whether prediction criteria are satisfied and/or false prediction criteria are satisfied.

In some cases, server 94 may be configured to provide a secure storage site for data that has been collected from IMD 10, patient device(s) 6, and/or external device 12. In some cases, server 94 may assemble data in web pages or other documents for viewing by trained professionals, such as physicians and clinicians, via computing devices 99. One or more aspects of the illustrated system of FIG. 4 may be implemented with general network technology and functionality, which may be similar to that provided by the Medtronic CareLink® Network.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

As part of a remote monitoring operation for patient 4, server 94 may transmit the instructions for medical intervention to one or more computing devices 99 located with a physician of patient 4 or a caregiver of patient 4. For example, such instructions for medical intervention may include an instruction to change a drug dosage, timing, or selection, to schedule a visit with the clinician, or to seek medical attention. In further examples, server 94 may generate an alert to communicate to patient device 6 for presentation to patient 4. The alert may be based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In some examples, one or more of computing devices 99 may be a tablet or other smart device located with a clinician, by which the clinician may program, receive alerts from, and/or interrogate IMD 10. For example, the clinician may access various patient data including instrument measurements, sensor data, physiological parameters of IMD 10, electrocardiogram, and/or indications and/or indications of patient health collected by IMD 10 through a computing device 99, such as when patient 4 is in in between clinician visits, to check on a status of a medical condition.

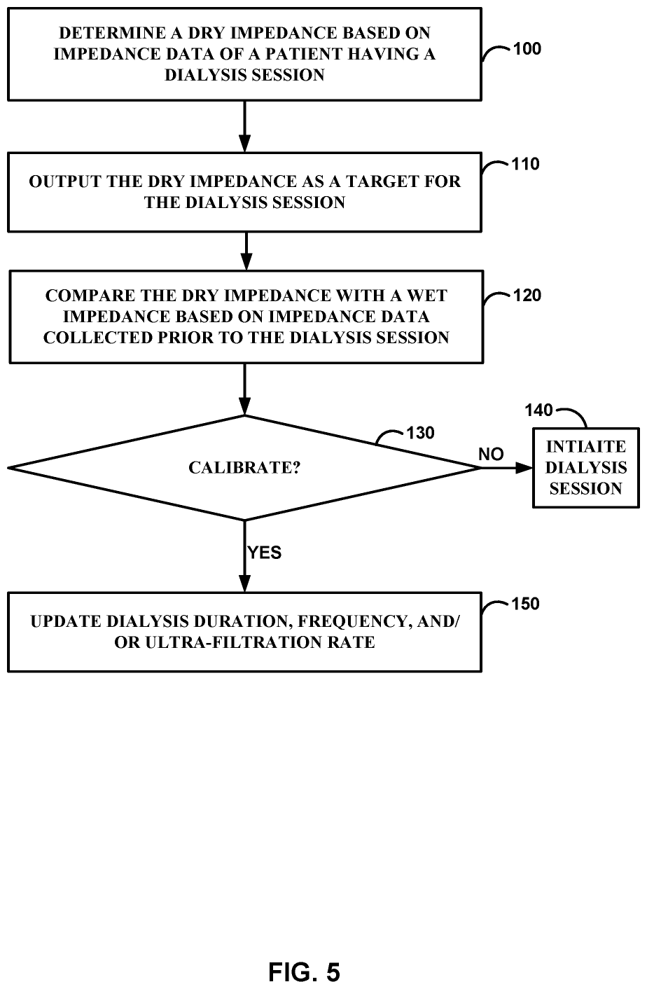

FIG. 5 is a flow diagram of an example operation for impedance-based dialysis control and/or calibration, in accordance with one or more examples of the present disclosure. In some examples, the example operation may be practiced by a computing device, such as external device 12 of FIGS. 1A-1B, of a physician with a patient undergoing dialysis via a dialysis system, such as dialysis system 14 of FIG. 1A.

The computing device, having access to impedance measurements from a medical device, such as IMD 10 of FIG. 1A, may facilitate successful dialytic treatments for the patient, such as patient 4 of FIG. 1A-1B, by computing an accurate fluid volume estimate of the patient's body prior to and/or (e.g., immediately) after a dialysis session, and (if needed) calibrating the dialysis system, such as dialysis system 14 of FIG. 1A, to perform a therapeutic and safe procedure at that dialysis session and/or a next dialysis session; for at least this reason, the following describes the example operation with reference to these elements. It can be appreciated that the physician device may determine the fluid volume estimate at any time, including different times through a day/week, assuming (e.g., relevant) impedance data is available. More importantly, the physician device may determine a fluid volume estimate based on the impedance data collected by and retrieved from the medical device of the patient.

In the example operation of FIG. 5, processing circuitry of external device 12 determines a dry impedance based on impedance data of patient 4 having a dialysis session and one or more medical devices including IMD 10 (100). The processing circuitry of external device 12 may output the dry impedance for presentation to the physician and/or configuration of dialysis system 14, for example, by establishing the dry impedance as a target (e.g., criterion) for the dialysis session (110). To illustrate by way of example dialysis session, when patient 4 arrives at a medical facility to receive their dialytic treatment(s), processing circuitry of external device 12 may determine one or more fluid volume estimate parameters for the patient based on various impedance data. The impedance data relied upon to determine a current amount of fluid in the patient's body may include impedance measurements that have been collected/recorded during an intra-dialytic period, which covers any time during a current dialysis session, and/or an inter-dialytic period, which covers any time between a previous (e.g., prior) dialysis session and the current dialysis session. The patient may have their own personalized/customized dry impedance for that session and subject to change depending on day of week, arrhythmia or heart rate, amount of fluid estimated from impedance change during inter-dialytic period, and/or the like. The physician may accept the dry impedance for one or more subsequent dialysis sessions.

IMD 10 may record the impedance data used for the above-mentioned determination at any point-in-time before the current dialysis session and/or after the previous dialysis session. The dry impedance may be a measurement (e.g., value (in ohms)) determined from impedance data collected during and/or after the previous dialysis session. The processing circuitry of external device 12 may implement a mechanism (e.g., a mathematical model such as a machine learning model) to predict accurate (bodily) fluid volume estimates corresponding to one or more (e.g., historical and/or contemporaneous) impedance measurements. One example model may define, for the current dialysis session of patient 4, a function that generates, as output, the dry impedance for patient 4 based on a number of model parameters (e.g., impedance-based features). This function may be configured to accept, as input, some of the model parameters including impedance measurement(s) indicating an amount of fluid removed at the previous dialysis session, an amount of fluid remaining after the previous dialysis session, a (most) recent dry impedance, a current fluid volume estimate including an amount of fluid prior to the dialysis session, and/or the like. The most recent dry impedance may be a dry impedance measurement value recorded immediately after the previous dialysis session concluded. In one example, the dry impedance may be representative of a (e.g., target) dry weight for patient 4. In some examples, the above-mentioned model incorporates one or more additional/alternative parameters including, but not limited to, a day of the week, heart rate measurements, patient medical history from EHR 18 of FIG. 1A, and/or the like.

As illustrated in FIG. 5, the processing circuitry of external device 12 compares the dry impedance to a wet impedance based on impedance data collected prior to the dialysis session and/or after the previous dialysis session (120). The comparison may result in a new/updated dry impedance (e.g., target). Because the impedance measurements provide accurate fluid volume measurements, the processing circuitry of external device 12 may determine an amount of fluid in patient 4's body after some (if not a substantial amount) of fluid has been removed by the dialytic treatment(s). Combined with a current body weight, the determined amount of fluid may be used to determine a dry weight of patient 4. Therefore, external device 12 may utilize the dry impedance as a personalized dry impedance (e.g., target threshold or criterion) for initializing certain dialysis parameters for a next dialysis session. In some examples, the processing circuitry of external device 12 may memorialize the new/updated dry impedance computed from impedance measurements that were recorded after successful completion of the current dialysis session

Similarly, the processing circuitry of external device 12 may have determined the dry impedance after completion of the dialysis session; since the previous dry impedance is available/known, the processing circuitry of external device 12 may establish that impedance value as the target for the current dialysis session and/or the next dialysis session. If the dry impedance from the previous session is unavailable/unknown, the wet impedance may be used to estimate the dry impedance for the current dialysis session. Alternatively, the dry impedance may be set to a default value for at least an initial dialysis session. Therefore, external device 12 may utilize the dry impedance of the previous dialysis session as a personalized dry impedance (e.g., target threshold or criterion) for initializing certain parameters for the current dialysis session.

The physician may prescribe patient 4 to a calibration period at a beginning of the current dialysis session. For patient 4 about to undergo the dialysis session, the processing circuitry of external device 12 may further personalize the dry impedance estimate depending on various additional factors, such as implant location tissue type around implant (for ICM), seasonal weather (humidity and sweating), and/or the like, during the calibration period.

During the calibration period, a risk assessment for adverse events may be performed. An example mechanism (e.g., machine learning model) for performing the risk assessment may be based on multiple factors (e.g., model parameters), which may fluctuate from a start of a dialysis session to an end of the session, and configured to generate, as output, a risk level (e.g., a quality) indicative of a probability (e.g., a likelihood) of an adverse effect occurring from patient 4 undergoing the dialysis session. The machine learning model may accept, as input, a combination of patient physiological parameters including, but not limited to time-stamped measurements for heart rate (HR), heart rate variability (HRV), blood pressure, fluid volumes in patient 4, and/or the like. Another set of parameters may incorporate data from patient 4's medical history including an adverse medical history such as an arrhythmia history, HF history, and/or the like. At the start of the dialysis session, the processing circuitry of external device 12 may compute another risk level (e.g., of a specific adverse event occurring due to (e.g., treatment from) the (current) dialysis session). This risk level/probability may be more fine-grained/acute and/or based on additional patient physiological parameters, such as an amount of fluid (e.g., fluid volume) from impedance data, incidence of arrhythmia since the previous dialysis session, a current blood pressure, different electrolyte levels, heart rate, respiratory rate, temperature, current hydration level, and/or other physiological parameters.

During the dialysis session, the processing circuitry of external device 12 may compute an example acute risk level based on (e.g., session-based) change(s) in impedance measurements, change(s) in heart rate, change(s) in respiratory rate, change(s) in temperature, change(s) in hydration, and/or the like. The acute risk level may be used to calibrate one or more of the dialysis parameters, including when to stop the dialytic treatment (i.e., duration). The risk level may prompt calibration of parameters for a future (e.g., next) dialysis session on that patient. The acute risk level may reflect a short-term risk assessment for a duration of the dialysis session. For that session and/or a next dialysis session, external device 12 may determine a probability for one or more specific adverse effects occurring in patient 4. Examples of these adverse effects include a hydration drop, an abnormal blood pressure (e.g., hypertension or hypotension), and/or the like. Additional sensor data (e.g., blood pressure measurements for that day and other short term physiological data) may enhance an accuracy of the acute risk-level/short-term risk assessment.

According to FIG. 5, the processing circuitry of external device 12 determines whether to calibrate the dialysis session (130). Based on a determination not to adjust the treatment of the dialysis session (NO of 130), the processing circuitry may proceed to apply a current dialytic treatment originally planned for patient 4 (140). Multiple parameters may define this treatment such that if the processing circuitry of external device 12 determines that the wet impedance of patient 4 satisfies the dry impedance target, the processing circuitry of external device 12 refrains from substantially changing any one or more dialysis parameters for controlling performance of the next (now current) dialysis session. The same parameters used for the previous session should provide same or similar results.

Based on a determination to adjust the treatment of the next dialysis session (YES of 130), the processing circuitry may update one or more dialysis parameters, such as dialysis duration, frequency, and/or ultra-filtration rate (150). Once the current dialysis session commences, the processing circuitry of external device 12 detects one or more impedance changes in the recorded electrical activity; for instance, by continuously monitoring the electrical activity in patient 4's body, the processing circuitry of external device 12 may determine an accurate estimate for how much fluid has been removed from patient 4 and/or a trajectory of the impedance changes to determine whether the ultrafiltration rate, the duration, and/or the estimated dry impedance is to be calibrated.

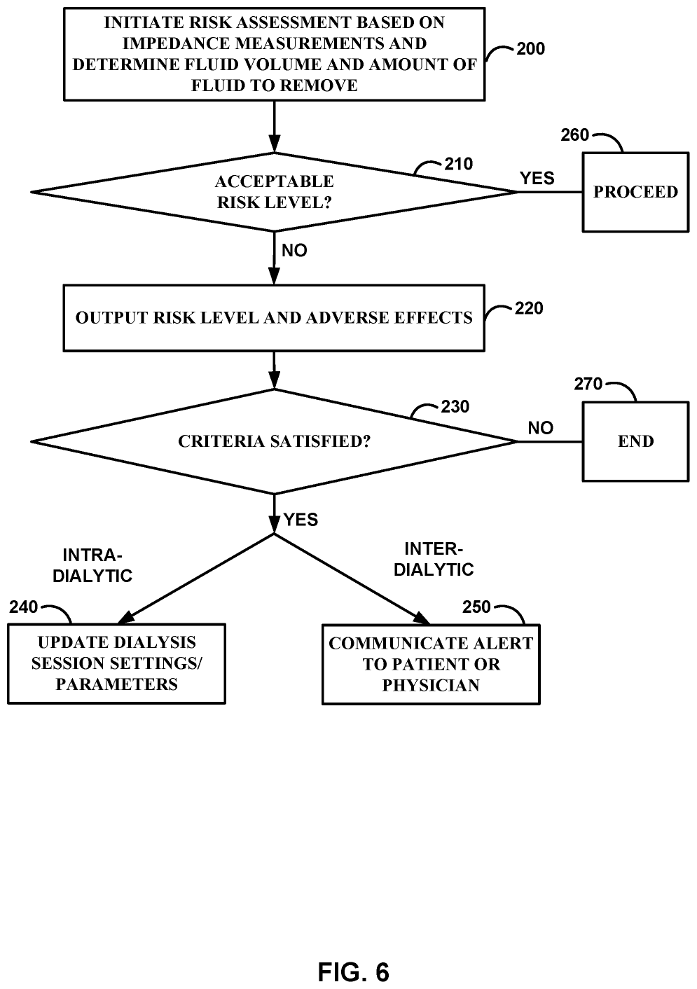

FIG. 6 is a flow diagram illustrating an example operation for monitoring a patient undergoing dialysis, in accordance with one or more examples of the present disclosure. In some examples, the example operation may be implemented for a computing device, such as patient device 6 and/or external device 12 of FIGS. 1A-1B, of a dialysis session for a patient. The computing device, having access to impedance measurements from a medical device, such as IMD 10 of FIG. 1A, of the patient, may facilitate a number of benefits for the patient and their health; for at least this reason, the following describes the example operation with reference to these elements.

According to the illustrated example of FIG. 6, processing circuitry of medical system 2 initiates a risk assessment based on one or more diagnostic factors, including impedance measurements, and determines a fluid volume (e.g., of the blood) (200). The patient may benefit, in general, from better medical care, but also at least from an accurate risk assessment. The risk assessment may be preventative of adverse effects from the dialysis session but also ongoing (if desired) with remote patient monitoring to further prevent patient harm (e.g., from high blood pressure). As described herein, the risk assessment is part (i.e., intra-dialytic) of or in preparation (e.g., inter-dialytic) of a dialysis session, which is a medical procedure to remove fluid from a human body performed by equipment represented by dialysis system 14 of FIG. 1A.

In the example operation of FIG. 6, the processing circuitry determines whether there is an acceptable risk level to the patient based on the one or more diagnostics (210). The risk level results from the above risk assessment and is indicative of a probability (e.g., likelihood) of one or more adverse effects occurring given the impedance measurements. The one or more adverse effects refer to a generic adverse effect or a specific effect (e.g., heart failure). There are number of factors that can be used to render the determination. Based on the determination that the risk level is acceptable (YES of 210), the processing circuitry may allow a next dialysis session to proceed (260). The next dialysis session may occur immediately after the risk assessment (e.g., intra-dialytic period) or may be scheduled for a later date (e.g., inter-dialytic period). There may be a calibration period when the next dialysis session is pending to ensure the dialysis system is properly configured based on patient impedance data.

Based on the determination that the risk level is unacceptable (NO of 210), the processing circuitry may output, via a display device, the one or more adverse effects and determine an amount of fluid to remove at the next dialysis session (220). The processing circuitry may proceed to determine satisfaction of certain criteria (230) and based on that determination that the certain criteria are not satisfied (NO of 230), the processing circuitry may end or terminate the next dialysis session for being unsafe and potentially harmful to the patient (270). Hence, the criteria may be configured to decide if it is within the patient's best medical interests to undergo the next dialysis session. For a patient who needs dialysis, the processing circuitry may allow the next dialysis session to proceed in which case the processing circuitry may change one or more dialysis parameters to reduce the patient's risk level, such as an ultra-filtration rate or a dialysis duration.

Based on that determination that the certain criteria are satisfied (YES of 230), the processing circuitry may proceed to a next action depending on whether a current time falls into the intra-dialytic period or the inter-dialytic period. In some examples, processing circuitry 80 of external device 12, being operated by a physician, initiates the risk assessment prior to the dialysis session. While communicatively coupled to dialysis system 14, processing circuitry 80 may determine that the physician initiated the risk assessment (e.g., at a hospital) (INTRA-DIALYTIC of 230) and for at least that reason, proceeds to update dialysis session settings/parameters to adjust the performance of the next dialysis session (240). In some examples, the physician may initiate the update for the dialysis session settings/parameters based on a risk level. In other examples, dialysis system 14 performs an automated risk assessment in which case updating, based on the risk level, the dialysis session settings/parameters may be automatic.

In other examples, processing circuitry 50 of IMD 10 (and/or processing circuitry of patient device 6) may coordinate patient monitoring operations, for example, in cooperation with one or more remote computing devices of computing system 20. While communicatively coupled to each other, these devices may monitor patient data and generate alert if the patient data indicates a non-trivial decline in patient health (e.g., a likely occurrence of a malady such as an arrythmia). To illustrate by way of example, while continuously monitor impedance measurements and communicatively coupled with patient device 6, processing circuitry 50 of IMD 10 may, at some point, determine that the impedance measurements indicate poor patient health, such as high blood pressure (INTER-DIALYTIC of 230) and for at least that reason, communicate an alert for display to the patient via patient device 6 or for display to the physician via external device 12 and/or dialysis system 14 (250).

In an example where IMD 10 is equipped with appropriate therapy delivery circuitry, IMD 10 may proceed to provide some therapy (if appropriate). One example therapy that IMD 10 may deliver includes cardioversion or defibrillation shock with the aid of an output circuit that determines whether a monophasic or biphasic pulse is delivered, whether housing electrode serves as cathode or anode, and which electrodes are involved in delivery of the cardioversion or defibrillation pulses. Such functionality may be provided by one or more switches or a switching circuitry of the therapy delivery circuitry.

The order and flow of the operation illustrated in FIGS. 5-6 is an example. In other examples according to this disclosure, more or fewer thresholds may be considered. Further, in some examples, processing circuitry may perform or not perform the method of FIG. 5 or 6, or any of the techniques described herein, as directed by a user, e.g., via external device 12 or computing devices 99. For example, a patient, clinician, or other user may turn on or off functionality for identifying changes in patient health (e.g., using Wi-Fi or cellular services) or locally (e.g., using an application provided on a patient's cellular phone or using a medical device programmer).

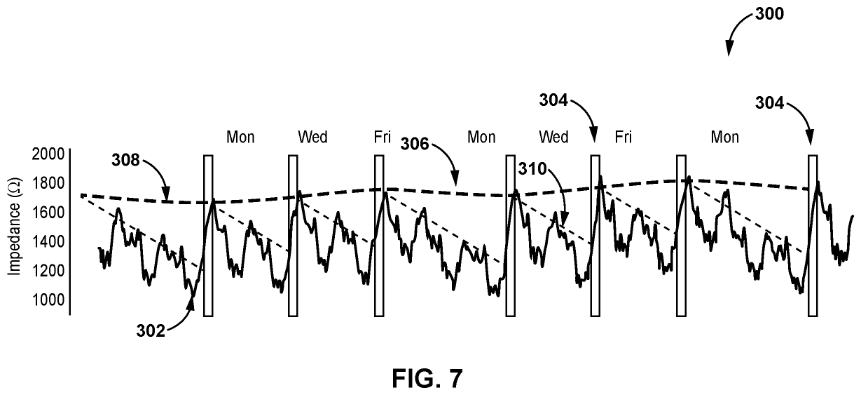

FIG. 7 is a chart 300 illustrating dynamic dialysis calibration according to the techniques described herein. Chart 300 may depict a series of (e.g., continuous) impedance measurements 302 being recorded by a medical device over a number of days. An amount of time tracked by chart 300 may be partitioned into intra-dialytic period(s) 304 (e.g., a time period in which a dialysis session is occurring or is about to occur) and inter-dialytic period(s) 306 (e.g., a time period between dialysis sessions in which no dialysis session is occurring or is about to occur), only some of which are labeled in FIG. 7 for ease of illustration.

The physician, via a computing device (e.g., external device 12 of FIG. 1A), may program the medical device to record the impedance measurements according to a schedule. In this manner, the medical device may record measurements for chart 300 at any resolution. In one example, the computing device generates chart 300 with an example resolution set to record the impedance measurements of the medical device every hour during an inter-dialysis period and/or every 5-minute during an intra-dialysis period. The computing device may display chart 300 to help the physician manage the dialysis session for the patient.

As depicted in chart 300, an estimated (target) dry impedance 308 may be determined based on impedance data as a more accurate replacement for fluid volume. One example use of the impedance data may be to determine an estimated dry impedance 302 as representative of a duration or a time limit indicating when to conclude the dialysis session. In this manner, the estimated dry impedance 302 may function as a personalized or calibrated target impedance value/level for the patient. The estimated dry impedance for any point-in-time may be based on fluid accumulation estimates from previous impedance measurements and possibly, other factors including a day of week and other diagnostic information. As the medical device continuously records impedance measurements at the example resolution for the remainder of the intra-dialysis period, the computing device may proceed to update estimated dry impedance 308. These continuous measurements may be used to estimate body fluid composition or a state of hydration to determine when to end the dialysis session or change dialysis parameters as a close loop control mechanism.

After the dialysis session and/or in preparation of a next dialysis session (e.g., inter-dialytic period), the medical device may continue to record impedance measurements at the above example resolution or another resolution. These measurements may be used to compute wet impedance values 310 and the dialysis parameters for the next dialysis session. One example dialysis parameter may be an ultra-filtration rate and/or a duration computed from the wet impedance and target dry impedance values, the day of week, and/or other diagnostic factors.

To generate chart 300, the medical device may sense an impedance of a fluid (e.g., blood plasma) and record that impedance measurement at various points-in-time during a day. One or more impedance measurements may indicate fluid (e.g., plasma) volume at one or more corresponding points-in-time. Therefore, a computing device performing the dynamic dialysis calibration may leverage the one or more recorded impedance measurements for use as a surrogate of fluid (e.g., plasma) volume. The medical device may record respective impedance measurements from any one or more of a number of spaces (e.g., body cavities), such as in intravascular space and/or extravascular or interstitial space. In some examples, the medical device may include IPG, leadless pacemaker devices, implantable cardioverter-defibrillator (ICD) or CRT devices in which electrodes of intracardiac leads measure impedance (e.g., in ohms) while capturing electrical activity (e.g., signals) in one or more locations (e.g., within the intravascular space). Therefore, each lead in the intravascular space may be configured to sense the electrical activity from a different position on a heart. In other examples, the medical device may include an insertable cardiac monitor (ICM), an extravascular (EV) implantable cardioverter-defibrillator (ICD), a subcutaneous ICD, and/or cutaneous patches in which electrodes of extravascular leads measure impedance while capturing electrical activity (e.g., signals) in one or more locations within the extravascular space (e.g., in substernal space). In one example, the ICM may be combined with the ICD into a unitary medical device where the patient benefits from extravascular impedance monitoring and pacing therapies such as anti-tachycardia pacing. In another example, the medical device may be configured to record impedance measurements from a combination of an interstitial space (within the extravascular space) and intravascular space in a volume measurement vector. Hence, multiple devices (including non-medical devices) may be used to provide impedance measures and (possibly) orthogonal information about fluid(s) in different fluid compartments in the body. As an alternative to the above example medical devices, a galvanometer (e.g., EKG machine) may be configured to record the impedance measurements.

In some examples, the dynamic dialysis calibration is integrated into remote patient monitoring operations by computing system 20. Should current patient activity indicate a substantial likelihood of an adverse effect occurring from the next dialysis, the medical device may communicate an alert to computing system 20. In turn, computing system 20 performs a number of monitoring operations to at least inform the patient and/or their physician of unhealthy patient activity. To illustrate by way of an example, the above-mentioned estimated dry impedance may be incorporated into a heart failure (HF) detection algorithm, for instance, by replacing or supplementing the impedance scores used in computing HF risk scores. For example, IMD 10 and/or external device 12 of FIG. 1A may replace a fixed impedance threshold (e.g., 600 ohms) for applications of the HF detection algorithm (e.g., TRIAGEHF™ algorithm, from Medtronic, Inc.) with the estimated (target) dry impedance, thereby operating as a patient-specific impedance threshold.

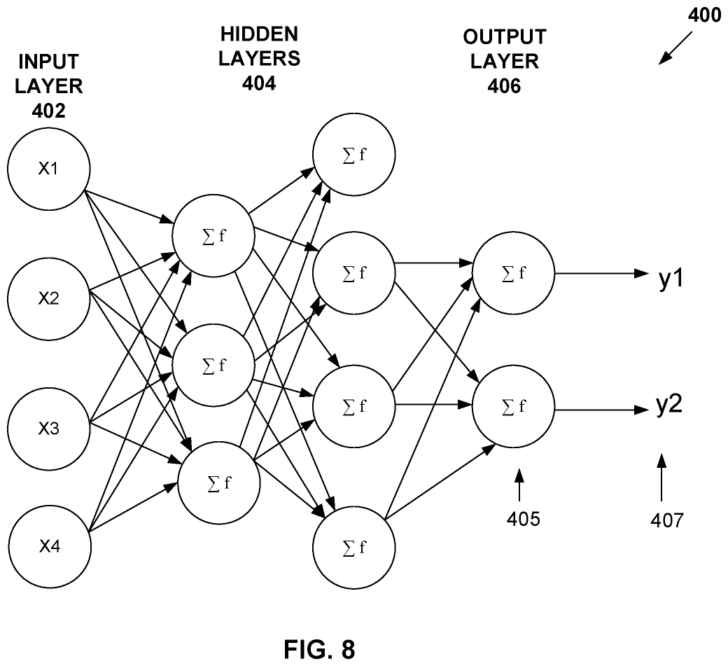

FIG. 8 is a conceptual diagram illustrating an example machine learning model 400 configured to determine one or more of dialysis parameters, target dry weight, fluid volume, or risk of a dialysis session based on impedance measurements and, in some cases, other physiological parameter values. Machine learning model 400 is an example of a deep learning model, or deep learning algorithm. One or more of IMD 10, computing devices 6, external device 12, or computing system 20 may train, store, and/or utilize machine learning model 300, but other devices may apply inputs associated with a particular patient to machine learning model 400 in other examples. Some non-limiting examples of machine learning techniques include Bayesian probability models, Support Vector Machines, K-Nearest Neighbor algorithms, and Multi-layer Perceptron.

As shown in the example of FIG. 8, machine learning model 400 may include three layers. These three layers include input layer 402, hidden layer 404, and output layer 406. Output layer 406 comprises the output from the transfer function 405 of output layer 406. Input layer 402 represents each of the input values X1 through X4 provided to machine learning model 400. The number of inputs may be less than or greater than 4, including much greater than 4, e.g., hundreds or thousands. In some examples, the input values may be any of the of values described above. In some examples, input values may include parameter values described herein, e.g., impedance measurements, morphological or other parameters of the ECG or heart sounds, heart rate variability, and arrhythmia metrics.

Each of the input values for each node in the input layer 402 is provided to each node of hidden layer 404. In the example of FIG. 8, hidden layers 404 include two layers, one layer having four nodes and the other layer having three nodes, but fewer or greater number of nodes may be used in other examples. Each input from input layer 402 is multiplied by a weight and then summed at each node of hidden layers 404. During training of machine learning model 400, the weights for each input are adjusted to establish the relationship between the inputs determining a score indicative of whether a set of inputs may be representative of a particular risk level or health state suggestion certain dialysis parameters. In some examples, one hidden layer may be incorporated into machine learning model 400, or three or more hidden layers may be incorporated into machine learning model 400, where each layer includes the same or different number of nodes.

The result of each node within hidden layers 404 is applied to the transfer function of output layer 406. The transfer function may be liner or non-linear, depending on the number of layers within machine learning model 400. Example non-linear transfer functions may be a sigmoid function or a rectifier function. The output 407 of the transfer function may be or a score indicative of a risk level (e.g., likelihood or probability) of an adverse health event due to a dialysis session. By applying the patient parameter data to a machine learning model, such as machine learning model 400, processing circuitry of system 2 is able to determine risk of and/or parameters for dialysis great accuracy, specificity, and sensitivity. This may facilitate improved therapy efficacy and safety, and improved patient health.

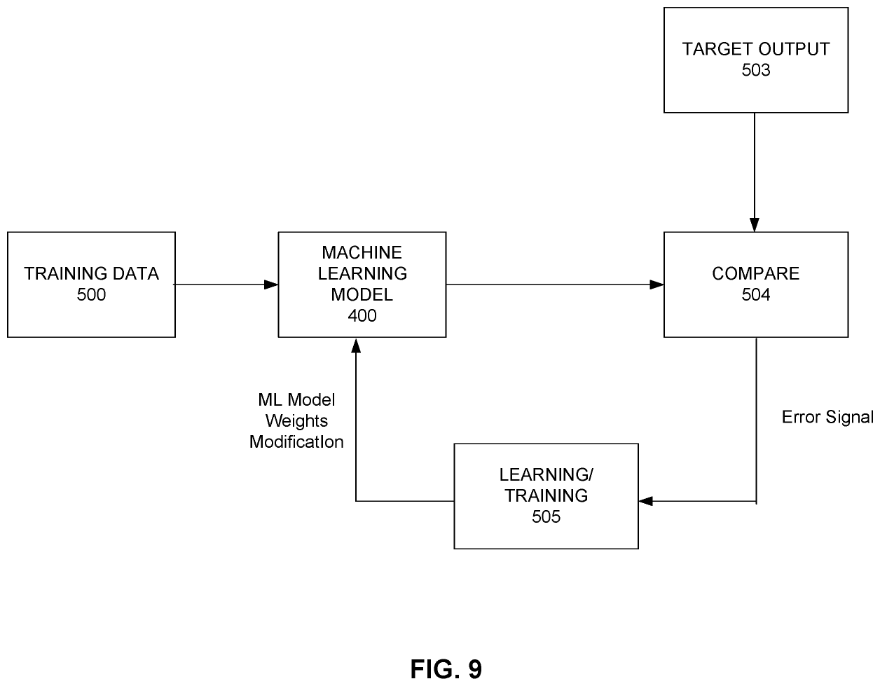

FIG. 9 is an example of a machine learning model 400 being trained using supervised and/or reinforcement learning techniques. Machine learning model 400 may be implemented using any number of models for supervised and/or reinforcement learning, such as but not limited to, an artificial neural network, a decision tree, naïve Bayes network, support vector machine, or k-nearest neighbor model, to name only a few examples. In some examples, processing circuitry one or more of IMD 10, computing device 6, external device 12, and/or computing system 20 initially trains the machine learning model 400 based on training set data 500 including numerous instances of input data corresponding to various risk levels and/or other patient conditions. An output of the machine learning model 400 may be compared 504 to the target output 503, e.g., as determined based on the label. Based on an error signal representing the comparison, the processing circuitry implementing a learning/training function 505 may send or apply a modification to weights of machine learning model 400 or otherwise modify/update the machine learning model 400. For example, one or more of IMD 10, computing device 6, external device 12, and/or computing system 20 may, for each training instance in the training set 500, modify machine learning model 400 to change a score generated by the machine learning model 400 in response to data applied to the machine learning model 400.

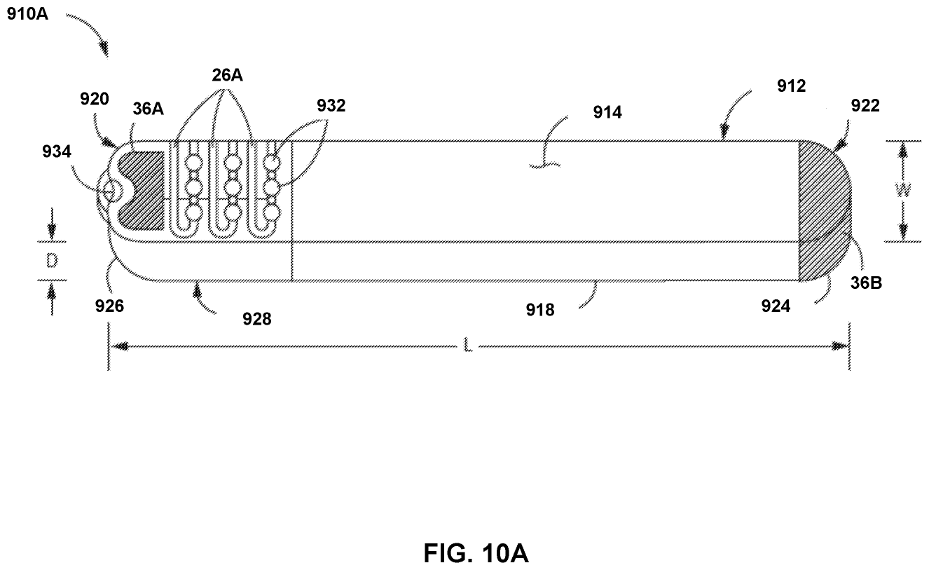

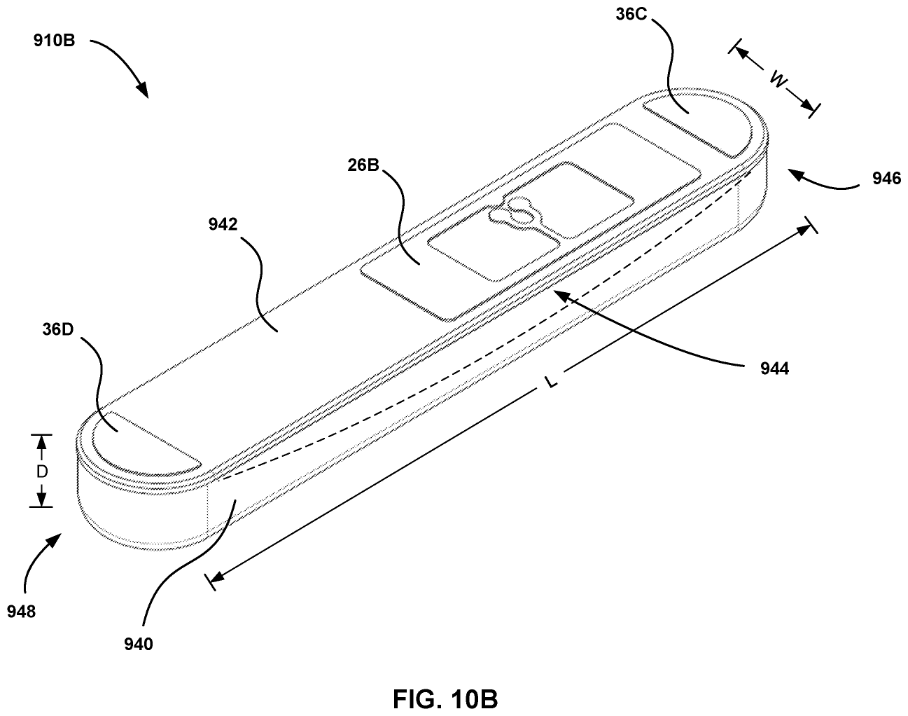

FIG. 10A is a perspective drawing illustrating an IMD 910A, which may be an example configuration of IMD 10 (e.g., IMD 10A) of FIGS. 1-3 as an ICM. In the example shown in FIG. 10A, IMD 910A may be embodied as a monitoring device having housing 912, proximal electrode 36A and distal electrode 36B. Housing 912 may further comprise first major surface 914, second major surface 918, proximal end 920, and distal end 922. Housing 912 encloses electronic circuitry located inside the IMD 910A and protects the circuitry contained therein from body fluids. Housing 912 may be hermetically sealed and configured for subcutaneous implantation. Electrical feedthroughs provide electrical connection of electrodes 36A and 36B.

In the example shown in FIG. 10A, IMD 910A is defined by a length L, a width W and thickness or depth D and is in the form of an elongated rectangular prism wherein the length L is much larger than the width W, which in turn is larger than the depth D. In one example, the geometry of the IMD 910A—in particular a width W greater than the depth D—is selected to allow IMD 910A to be inserted under the skin of the patient using a minimally invasive procedure and to remain in the desired orientation during insertion. For example, the device shown in FIG. 10A includes radial asymmetries (notably, the rectangular shape) along the longitudinal axis that maintains the device in the proper orientation following insertion. For example, the spacing between proximal electrode 36A and distal electrode 36B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 910A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 914 may range from 3 mm to 15, mm, from 3 mm to 10 mm, or from 5 mm to 15 mm, and may be any single or range of widths between 3 mm and 15 mm. The thickness of depth D of IMD 910A may range from 2 mm to 15 mm, from 2 mm to 9 mm, from 2 mm to 5 mm, from 5 mm to 15 mm, and may be any single or range of depths between 2 mm and 15 mm. In addition, IMD 910A according to an example of the present disclosure is has a geometry and size designed for ease of implant and patient comfort. Examples of IMD 910A described in this disclosure may have a volume of three cubic centimeters (cm) or less, 1.5 cubic cm or less or any volume between three and 1.5 cubic centimeters.

In the example shown in FIG. 10A, once inserted within the patient, the first major surface 914 faces outward, toward the skin of the patient while the second major surface 918 is located opposite the first major surface 914. In addition, in the example shown in FIG. 10A, proximal end 920 and distal end 922 are rounded to reduce discomfort and irritation to surrounding tissue once inserted under the skin of the patient. IMD 910A, including instrument and method for inserting IMD 910A is described, for example, in U.S. Patent Publication No. 2014/0276928, incorporated herein by reference in its entirety.

Proximal electrode 36A is at or proximate to proximal end 920, and distal electrode 36B is at or proximate to distal end 922. Proximal electrode 36A and distal electrode 36B are used to sense cardiac EGM signals, e.g., ECG signals, and measure interstitial impedance thoracically outside the ribcage, which may be sub-muscularly or subcutaneously. EGM signals and impedance measurements may be stored in a memory of IMD 910A, and data may be transmitted via integrated antenna 26A to another device, which may be another implantable device or an external device, such as computing device 6 and external device 12. In some example, electrodes 36A and 36B may additionally or alternatively be used for sensing any bio-potential signal of interest, which may be, for example, an EGM, EEG, EMG, or a nerve signal, from any implanted location. Housing 912 may house the circuitry of IMD 10 illustrated in FIG. 2.

In the example shown in FIG. 910A, proximal electrode 36A is at or in close proximity to the proximal end 920 and distal electrode 36B is at or in close proximity to distal end 922. In this example, distal electrode 36B is not limited to a flattened, outward facing surface, but may extend from first major surface 914 around rounded edges 924 and/or end surface 926 and onto the second major surface 918 so that the electrode 36B has a three-dimensional curved configuration. In some examples, electrode 36B is an uninsulated portion of a metallic, e.g., titanium, part of housing 912.

In the example shown in FIG. 10A, proximal electrode 36A is located on first major surface 914 and is substantially flat, and outward facing. However, in other examples proximal electrode 36A may utilize the three dimensional curved configuration of distal electrode 36B, providing a three dimensional proximal electrode (not shown in this example). Similarly, in other examples distal electrode 36B may utilize a substantially flat, outward facing electrode located on first major surface 914 similar to that shown with respect to proximal electrode 36A.

The various electrode configurations allow for configurations in which proximal electrode 36A and distal electrode 36B are located on both first major surface 914 and second major surface 918. In other configurations, such as that shown in FIG. 10A, only one of proximal electrode 36A and distal electrode 36B is located on both major surfaces 914 and 918, and in still other configurations both proximal electrode 36A and distal electrode 36B are located on one of the first major surface 914 or the second major surface 918 (e.g., proximal electrode 36A located on first major surface 914 while distal electrode 36B is located on second major surface 918). In another example, IMD 910A may include electrodes on both major surface 914 and 918 at or near the proximal and distal ends of the device, such that a total of four electrodes are included on IMD 910A. Electrodes 36A and 36B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 10A, proximal end 920 includes a header assembly 928 that includes one or more of proximal electrode 36A, integrated antenna 26A, anti-migration projections 932, and/or suture hole 934. Integrated antenna 26A is located on the same major surface (i.e., first major surface 914) as proximal electrode 36A and is also included as part of header assembly 928. Integrated antenna 26A allows IMD 910A to transmit and/or receive data. In other examples, integrated antenna 26A may be formed on the opposite major surface as proximal electrode 36A, or may be incorporated within the housing 912 of IMD 910A. In the example shown in FIG. 10A, anti-migration projections 932 are located adjacent to integrated antenna 26A and protrude away from first major surface 914 to prevent longitudinal movement of the device. In the example shown in FIG. 10A, anti-migration projections 932 include a plurality (e.g., nine) small bumps or protrusions extending away from first major surface 914. As discussed above, in other examples anti-migration projections 932 may be located on the opposite major surface as proximal electrode 36A and/or integrated antenna 26A. In addition, in the example shown in FIG. 10A, header assembly 928 includes suture hole 934, which provides another means of securing IMD 910A to the patient to prevent movement following insertion. In the example shown, suture hole 934 is located adjacent to proximal electrode 36A. In one example, header assembly 928 is a molded header assembly made from a polymeric or plastic material, which may be integrated or separable from the main portion of IMD 910A.

FIG. 10B is a perspective drawing illustrating another IMD 910B, which may be another example configuration of IMD 10 (e.g., IMD 10A) from FIGS. 1-3 as an ICM. IMD 910B of FIG. 10B may be configured substantially similarly to IMD 910A of FIG. 10A, with differences between them discussed herein.

IMD 910B may include a leadless, subcutaneously-implantable monitoring device, e.g. an ICM. IMD 910B includes housing having a base 940 and an insulative cover 942. Proximal electrode 36C and distal electrode 36D may be formed or placed on an outer surface of cover 942. Various circuitries and components of IMD 910B, e.g., described with respect to FIG. 3, may be formed or placed on an inner surface of cover 942, or within base 940. In some examples, a battery or other power source of IMD 910B may be included within base 940. In the illustrated example, antenna 26B is formed or placed on the outer surface of cover 942, but may be formed or placed on the inner surface in some examples. In some examples, insulative cover 942 may be positioned over an open base 940 such that base 940 and cover 942 enclose the circuitries and other components and protect them from fluids such as body fluids. The housing including base 940 and insulative cover 942 may be hermetically sealed and configured for subcutaneous implantation.

Circuitries and components may be formed on the inner side of insulative cover 942, such as by using flip-chip technology. Insulative cover 942 may be flipped onto a base 940. When flipped and placed onto base 940, the components of IMD 910B formed on the inner side of insulative cover 942 may be positioned in a gap 944 defined by base 940. Electrodes 36C and 36D and antenna 26B may be electrically connected to circuitry formed on the inner side of insulative cover 942 through one or more vias (not shown) formed through insulative cover 942. Insulative cover 942 may be formed of sapphire (i.e., corundum), glass, parylene, and/or any other suitable insulating material. Base 940 may be formed from titanium or any other suitable material (e.g., a biocompatible material). Electrodes 36C and 36D may be formed from any of stainless steel, titanium, platinum, iridium, or alloys thereof. In addition, electrodes 36C and 36D may be coated with a material such as titanium nitride or fractal titanium nitride, although other suitable materials and coatings for such electrodes may be used.

In the example shown in FIG. 10B, the housing of IMD 910B defines a length L, a width W and thickness or depth D and is in the form of an elongated rectangular prism wherein the length L is much larger than the width W, which in turn is larger than the depth D, similar to IMD 910A of FIG. 10A. For example, the spacing between proximal electrode 36C and distal electrode 36D may range from 5 mm to 50 mm, from 30 mm to 50 mm, from 35 mm to 45 mm, and may be any single spacing or range of spacings from 5 mm to 50 mm, such as approximately 40 mm. In addition, IMD 910B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 910B may have a volume of three cubic centimeters (cm) or less, or 1.5 cubic cm or less, such as approximately 1.4 cubic cm.

In the example shown in FIG. 10B, once inserted subcutaneously within the patient, outer surface of cover 942 faces outward, toward the skin of the patient. In addition, as shown in FIG. 10B, proximal end 946 and distal end 948 are rounded to reduce discomfort and irritation to surrounding tissue once inserted under the skin of the patient. In addition, edges of IMD 910B may be rounded.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

The following examples are illustrative of the techniques described herein.

Example 1. A medical system comprising: one or more medical devices configured to collect physiological parameter data of a patient, wherein the physiological parameter data comprises impedance data corresponding to at least one fluid volume in the patient; and processing circuitry configured to: determine a dry impedance for the patient having a dialysis session based on the physiological parameter data; and generate output data indicating the dry impedance for presentation to a clinician.

Example 2. The medical system of example 1, wherein to determine the dry impedance, the processing circuitry is configured to: determine an estimate of the dry impedance based on impedance data collected at least one of during or after a previous dialysis session; and compare the dry impedance to at least one of impedance data collected between the dialysis session and the previous dialysis session or impedance data collected during the dialysis session.

Example 3. The medical system of example 1, wherein the processing circuitry is configured to generate dialysis parameter data for controlling at least one of the dialysis session or a next dialysis session based on the dry impedance, wherein the dry impedance is established as a target for impedance data collected during the dialysis session.

Example 4. A medical system comprising: one or more medical devices configured to collect physiological parameter data of a patient, wherein the physiological parameter data comprises impedance data corresponding to at least one fluid volume in the patient; and processing circuitry configured to: determine a risk level to the patient from undergoing a dialysis session based on the physiological parameter data; and generate output data indicating the risk level for presentation to a clinician.

Example 5. The medical system of example 4, wherein the processing circuitry is configured to generate dialysis parameter data for controlling the dialysis session based on at least one of the physiological parameter data or the risk level.

Example 6. The medical system of example 5, wherein the physiological parameter data comprises physiological parameter data collected during the dialysis session, and the dialysis parameter data comprises updated dialysis parameter data.

Example 7. The medical system of example 5 or 6, wherein the dialysis parameter data comprises an amount of the fluid volume to remove in the dialysis session.

Example 8. The medical system of any one or more of examples 5 to 7, wherein the dialysis parameter data comprises at least one of a blood flow rate, fluid removal rate, an ultra-filtration rate, a duration of the dialysis session, or a temperature.

Example 9. The medical system of any one or more of examples 4 to 8, wherein to determine the risk level, the processing circuitry is configured to: determine a risk level of an adverse effect occurring in the patient from the dialysis session.

Example 10. The medical system of any one or more of examples 4 to 9, wherein to generate the output data, the processing circuitry is configured to: generate the output data based on a determination that the risk level satisfies at least one criterion.

Example 11. The medical system of any one or more of examples 4 to 10, wherein the physiological data comprises physiological data collected during an inter-dialytic period between the dialysis session and a prior dialysis session, and the processing circuitry is further configured to: determine an estimate of a dry impedance for the patient for the dialysis session based on the physiological parameter data.

Example 12. The medical system of example 11, wherein the processing circuitry is configured to at least one of: output the estimate of dry impedance for presentation to the clinician; or generate dialysis parameter data for controlling the dialysis session based on the estimate of dry impedance.

Example 13. The medical system of any one or more of examples 4 to 12, wherein the processing circuitry is configured to output the physiological parameter data for presentation to the clinician.

Example 14. The medical system of any one or more of examples 4 to 13, wherein the one or more medical devices are configured to collect impedance data at a first rate during an inter-dialytic period between the dialysis session and a prior dialysis session and a second rate during an intra-dialytic period during the dialysis session.

Example 15. The medical system of examples 13 and 14, wherein the processing circuitry is configured to output the physiological parameter data for presentation substantially continuously during the intra-dialytic period.

Example 16. The medical system of any one or more of examples 4 to 15, wherein the impedance data for at least one fluid volume comprises impedance measurements for at least one of an extravascular space, an intravascular space, or an interstitial space.

Example 17. The medical system of example 16, wherein the processing circuitry is further configured to: determine a net filtration rate based on impedance measurements for the interstitial space and impedance measurements for the intravascular space for at least one of an inter-dialytic period between the dialysis session and a prior dialysis session or an intra-dialytic period during the dialysis session.

Example 18. The medical system of example 17, wherein the one or more medical devices comprise a first medical device configured to measure impedance for the interstitial space and a second medical device configured to measure impedance for the intravascular space.

Example 19. The medical system of example 17 or 18, wherein the one or more medical devices comprise an insertable cardiac monitor configured to measure impedance for the interstitial space.

Example 20. The medical system of example 16, wherein the processing circuitry is further configured to: determine a net filtration rate based on impedance measurements for the interstitial space and the physiological parameter data of another physiological parameter indicative of fluid volume in the intravascular space.

Example 21. The medical system of example 20, wherein the another physiological parameter comprises at least one of an electrocardiogram morphology parameter or a heart sound parameter.

Example 22. The medical system of any of examples 17 to 21, wherein the processing circuitry is configured to at least one of: output the net filtration rate for presentation to the clinician; determine the risk level based on the net filtration rate; or generate dialysis parameter data for controlling the dialysis session based on net filtration rate.

Example 23. The medical system of any of examples 1 to 22, wherein the physiological parameter data further comprises electrocardiogram data, tachyarrhythmia data, heart sound data, activity data, and posture data.

Example 24. A method performed by processing circuitry of a medical system having one or more medical devices configured to collect physiological parameter data of a patient, the method comprising: determining a risk level to the patient from undergoing a dialysis session based on the physiological parameter data, wherein the physiological parameter data comprises impedance data corresponding to at least one fluid volume in the patient; and generating output data indicating the risk level for presentation to a clinician.

Example 25. The method of example 24 further comprising: generating dialysis parameter data for controlling the dialysis session based on at least one of the physiological parameter data or the risk level.

Example 26. The method of example 24, wherein the physiological parameter data comprises physiological parameter data collected during the dialysis session, and the dialysis parameter data comprises updated dialysis parameter data.

Example 27. The method of example 24 or 25, wherein the dialysis parameter data comprises an amount of the fluid volume to remove in the dialysis session.

Example 28. The method of any one or more of examples 25 to 27, wherein the dialysis parameter data comprises at least one of a fluid rate, an ultrafiltration rate, or a duration of the dialysis session.

Example 29. The method of any one or more of examples 24 to 28, wherein determining the risk level further comprises: determining a risk level of an adverse effect occurring in the patient from the dialysis session.

Example 30. The method of any one or more of examples 24 to 29, wherein generating the output data further comprises: generating the output data based on a determination that the risk level satisfies at least one criterion.

Example 31. The method of any one or more of examples 24 to 30, wherein the physiological data comprises physiological data collected during an inter-dialytic period between the dialysis session and a prior dialysis session, the method further comprising: determining an estimate of a dry impedance for the patient for the dialysis session based on the physiological parameter data.

Example 32. The method of example 31 further comprising at least one of: outputting the estimate of dry impedance for presentation to the clinician; or generating dialysis parameter data for controlling the dialysis session based on the estimate of dry impedance.

Example 33. The method of any one or more of examples 24 to 32 further comprising: outputting the physiological parameter data for presentation to the clinician.

Example 34. The method of any one or more of examples 24 to 33, wherein the one or more medical devices are configured to collect impedance data at a first rate during an inter-dialytic period between the dialysis session and a prior dialysis session and a second rate during an intra-dialytic period during the dialysis session.

Example 35. The method of example 33 or 34 further comprising: outputting the physiological parameter data for presentation substantially continuously during the intra-dialytic period.

Example 36. The method of any one or more of examples 34 to 35, wherein the impedance data for at least one fluid volume comprises impedance measurements for at least one of an extravascular space, an intravascular space, or an interstitial space.

Example 37. The method of example 36, wherein the processing circuitry is further configured to: determine a net filtration rate based on impedance measurements for the interstitial space and impedance measurements for the intravascular space for at least one of an inter-dialytic period between the dialysis session and a prior dialysis session or an intra-dialytic period during the dialysis session.

Example 38. The method of example 37, wherein the one or more medical devices comprise a first medical device configured to measure impedance for the interstitial space and a second medical device configured to measure impedance for the intravascular space.

Example 39. The method of example 37 or 38, wherein the one or more medical devices comprise an insertable cardiac monitor configured to measure impedance for the interstitial space.

Example 40. The method of example 36, wherein the processing circuitry is further configured to: determine a net filtration rate based on impedance measurements for the interstitial space and the physiological parameter data of another physiological parameter indicative of fluid volume in the intravascular space.

Example 41. The method of example 40, wherein the another physiological parameter comprises at least one of an electrocardiogram morphology parameter or a heart sound parameter.

Example 42. The method of any of examples 37 to 41, wherein the processing circuitry is configured to at least one of: output the net filtration rate for presentation to the clinician; determine the risk level based on the net filtration rate; or generate dialysis parameter data for controlling the dialysis session based on net filtration rate.

Example 43. The method of any of examples 24 to 42, wherein the physiological parameter data further comprises electrocardiogram data, tachyarrhythmia data, heart sound data, activity data, and posture data.

Example 44. A method comprising steps performed by any medical system of examples 4-23.

Example 45. A computing device configured to perform any method of examples 24-43.

Example 46. A non-transitory computer-readable storage medium comprising program instructions that, when executed by processing circuitry of a medical system, cause the processing circuitry to perform each step of any method of examples 24-43.

1-19. (canceled)

20. A medical system comprising:
one or more medical devices configured to collect physiological parameter data of a patient, wherein the physiological parameter data comprises impedance data corresponding to at least one fluid volume in the patient, and the impedance data for at least one fluid volume comprises impedance measurements for at least two of an extravascular space, an intravascular space, or an interstitial space; and
processing circuitry configured to:
determine a dry impedance for the patient having a dialysis session based on the physiological parameter data; and
generate output data indicating the dry impedance for presentation to a clinician.

21. The medical system of claim 20, wherein to determine the dry impedance, the processing circuitry is configured to:
determine an estimate of the dry impedance based on impedance data collected at least one of during or after a previous dialysis session; and
compare the dry impedance to at least one of impedance data collected between the dialysis session and the previous dialysis session or impedance data collected during the dialysis session.

22. The medical system of claim 20, wherein the processing circuitry is configured to generate dialysis parameter data for controlling at least one of the dialysis session or a next dialysis session based on the dry impedance, wherein the dry impedance is established as a target for impedance data collected during the dialysis session.

23. The medical system of claim 20, wherein the processing circuitry is configured to:
determine a risk level to the patient from undergoing a dialysis session based on the physiological parameter data; and
generate output data indicating the risk level for presentation to the clinician.

24. The medical system of claim 23, wherein the processing circuitry is configured to generate dialysis parameter data for controlling the dialysis session based on the risk level.

25. The medical system of claim 24, wherein the physiological parameter data comprises physiological parameter data collected during the dialysis session, and the dialysis parameter data comprises updated dialysis parameter data.

26. The medical system of claim 24, wherein the dialysis parameter data comprises an amount of the fluid volume to remove in the dialysis session.

27. The medical system of claim 24, wherein the dialysis parameter data comprises at least one of a blood flow rate, fluid removal rate, an ultra-filtration rate, a duration of the dialysis session, or a temperature.

28. The medical system of claim 23, wherein to determine the risk level, the processing circuitry is configured to:
determine a risk level of an adverse effect occurring in the patient from the dialysis session.

29. The medical system of claim 23, wherein to generate the output data, the processing circuitry is configured to:
generate the output data based on a determination that the risk level satisfies at least one criterion.

30. The medical system of claim 20, wherein the one or more medical devices are configured to collect impedance data at a first rate during an inter-dialytic period between the dialysis session and a prior dialysis session and a second rate during an intra-dialytic period during the dialysis session.

31. The medical system of claim 20, wherein the processing circuitry is further configured to:
determine a net filtration rate based on impedance measurements for the interstitial space and impedance measurements for the intravascular space for at least one of an inter-dialytic period between the dialysis session and a prior dialysis session or an intra-dialytic period during the dialysis session.

32. The medical system of claim 20, wherein the one or more medical devices comprise a first medical device configured to measure impedance for the interstitial space and a second medical device configured to measure impedance for the intravascular space.

33. The medical system of claim 32, wherein the first medical device comprises an insertable cardiac monitor configured to measure impedance for the interstitial space.

34. The medical system of claim 33, wherein the insertable cardiac monitor comprises:
a housing configured for subcutaneous implantation in the patient, the housing having a length between 40 millimeters (mm) and 60 mm between a first end and a second end, a width less than the length, and a depth less than the width;
a first electrode at or proximate to the first end; and
a second electrode at or proximate to the second end,
wherein the insertable cardiac monitor is configured to measure impedance for the interstitial space via the first electrode and the second electrode.

35. The medical system of claim 20, wherein the processing circuitry is further configured to:
determine a net filtration rate based on impedance measurements for the interstitial space and the physiological parameter data of another physiological parameter indicative of fluid volume in the intravascular space.

36. The medical system of claim 35, wherein the another physiological parameter comprises at least one of an electrocardiogram morphology parameter or a heart sound parameter.

37. The medical system of claim 20, wherein the physiological parameter data further comprises electrocardiogram data, tachyarrhythmia data, heart sound data, activity data, and posture data.

38. A method performed by processing circuitry of a medical system having one or more medical devices configured to collect physiological parameter data of a patient, the method comprising:
determining a risk level to the patient from undergoing a dialysis session based on the physiological parameter data, wherein the physiological parameter data comprises impedance data corresponding to at least one fluid volume in the patient, and the impedance data for at least one fluid volume comprises impedance measurements for at least two of an extravascular space, an intravascular space, or an interstitial space; and
generating output data indicating the risk level for presentation to a clinician.

39. The method of claim 38, further comprising:
generating dialysis parameter data for controlling the dialysis session based on at least one of the physiological parameter data or the risk level.
